(19) United States
(12) Patent Application Publication
(10) Pub. No.: US 2016/0003837 A1
(43) Pub. Date: Jan. 7, 2016

(54) BIOMARKERS FOR THE PREDICTION OF PRETERM BIRTH

(71) Applicant: DUKE UNIVERSITY, Durham, NC (US)

(72) Inventors: Amy MURTHA, Hillsborough, NC (US); Liping FENG, Cary, NC (US)

(21) Appl. No.: 14/759,636

(22) PCT Filed: Jan. 8, 2014
(86) PCT No.: PCT/US14/10627

§ 371 Date: Jul. 7, 2015

Related U.S. Application Data
(60) Provisional application No. 61/750,063, filed on Jan. 8, 2013.

(51) Int. Cl. G01N 33/68; C12N 15/113; H01J 49/00; C12Q 1/68

(52) U.S. Cl. CPC G01N 33/689; C12Q 1/6883; C12N 15/1138; H01J 49/0027; G01N 2800/368; G01N 2333/705; C12Q 2600/158; C12Q 2600/106; G01N 2800/52; C12N 2310/14; C12N 2320/30

(57) ABSTRACT

The present disclosure provides biomarkers useful for determining the risk of, prognosis of, and/or diagnosis of conditions such as preterm birth in a subject.

### CROSS REFERENCE TO RELATED APPLICATIONS

This application is a 35 U.S.C. 371 national stage of International Patent Application No. PCT/US2014/010627 filed on Jan. 8, 2014, and claims the benefit of U.S. Provisional Patent Application No. 61/750,063 filed Janurary 8, 2013, the disclosure of which is hereby incorporated by reference in its entirety.

### TECHNICAL FIELD

The presently disclosed subject matter relates to biomarkers for predicting and monitoring preterm birth.

### BACKGROUND

Of the over 4 million births that occur in the US annually, 12.2%(over 500,000) occur preterm (prior to 37 weeks gestation). Despite strides in the care of the preterm infant little progress has been made on impacting preterm birth rates in the US. Preterm birth results in significant health care and societal costs estimated at 26 billion dollars annually. [1] In 2006, the Institute of Medicine issued a Consensus Report entitled “Preterm Birth: Causes, Consequences and Prevention”.[1] This report recommended a multidisciplinary research agenda aimed at improving the prediction and prevention of preterm birth and better understanding the health and developmental problems to which preterm infants are more vulnerable. Preterm birth is likely one of the most important factor predicting long term health. Compared to term infants, preterm infants have a greater risk of death and a variety of other health and developmental issues including long term cognitive, behavioral, social, emotional, neurodevelopmental and reproductive problems. [2, 3]

Traditionally, clinical research in preterm birth has focused on the treatment of the end stages of the disease process typically manifested by preterm contractions and preterm premature rupture of membranes. More recently the focus has been aimed at understanding the etiology of the disease process which likely begins early in pregnancy.

Development of potential biomarkers for preterm birth has been the focus of multiple investigations yet has been met with limited success. Recently the Preterm Birth International Collaborative (PREBIC) reviewed the existing literature on preterm birth biomarkers from the last 40 years. Of the 116 biomarkers reported, none emerged as a risk predictor for preterm birth. Several areas of concern over the existing studies emerged from this work including poor phenotype definition and study design and poor rationale for biomarker selection. [4] The majority of these investigations focus on the end stage inflammatory process which is either not relevant early in the disease process or once detected too late to have meaningful predictive abilities.

In clinical practice, the mainstays for preterm birth prediction are vaginal swab for fetal fibronectin and endovaginal ultrasound for cervical length. Multiple studies have shown that in symptomatic women, fetal fibronectin is able to predict preterm birth in about 35% of cases. Its use is most relevant when negative as its negative predictive value in the setting of symptoms approaches 95%. [5, 6] In contrast, cervical length measured by endovaginal ultrasound has the ability to predict preterm birth earlier in gestation in the absence of symptoms. [7-9] Endovaginal cervical length ultrasound is currently considered the most reliable predictor of preterm birth even in low risk women yet its clinical utility as a universal screening tool is currently the focus of much debate in obstetrics. Concerns over the quality and consistency of this measurement have limited the clinical use.[8]

Recent therapeutic attempts at reducing preterm birth rates has focused on two at risk groups; women with a history of preterm birth in a prior pregnancy and those with a short cervical length. Women with a history of preterm birth were evaluated in a large multicenter randomized trial which concluded that treatment of women with a history of spontaneous preterm birth treated with weekly intramuscular injection of 17 alpha hydroxyprogesterone caproate (17-OHPC) was associated with a 35% reduction in preterm birth rates. [10] This landmark study led to a shift in practice such that all women with a history of preterm birth are offered 17-OHPC during subsequent pregnancies. Importantly, screening based on obstetric history leaves out the important group of women having their first pregnancy. All pregnant women are potentially eligible for cervical length screening. Two large multinational trials have now demonstrated that vaginal progesterone reduces the risk of preterm birth by 45% in women identified as being at risk based on shortened cervical length.[11, 12] These two approaches are different in the patient population but often overlap. Based on these investigations it is clear that progesterone therapy likely plays an important role in preterm birth prevention. Key to the use of both vaginal progesterone and 17-OHPC is the identification of at risk individuals in which progesterone therapy will provide the most benefit.

Therefore, there is a pressing need to identify improved diagnostic and prognostic biomarkers for preterm birth.

### SUMMARY

The present disclosure provides methods of assessing the risk of a subject for entering preterm birth comprising, consisting of, or consisting essentially of quantifying the amount of at least one biomarker present in a biological sample derived from the subject, wherein the biomarker comprises, consists of, or consists essentially of a protein associated with preterm birth.

One aspect of the present disclosure provides a method of determining the risk of, prognosis of, and/or diagnosis of preterm birth in a subject comprising, consisting of, or consisting essentially of quantifying the amount of at least one biomarker present in a biological sample derived from the subject, wherein the biomarker is associated with preterm birth.

Another aspect of the present disclosure provides a method of diagnosing preterm birth in a subject comprising, consisting of, or consisting essentially of: obtaining a biological sample from a subject; determining the expression level of one or more biomarkers that are associated with preterm birth in the biological sample; and comparing the expression level of the biomarkers in the biological sample with that of a control, wherein the presence of one or more of the biomarkers in the sample that is in an amount greater than that of the control indicates preterm birth.

Another aspect of the present disclosure provides a method of determining the risk of a subject developing preterm birth comprising, consisting of, or consisting essentially of: obtaining a biological sample from a subject; determining the expression level of one or more biomarkers that are associated with preterm birth in the biological sample; and comparing the expression level of the biomarkers in the biological sample with that of a control, wherein the presence of one or more of the biomarkers in the sample that is in an amount greater than that of the control indicates preterm birth.

Another aspect of the present disclosure provides a method of determining the prognosis of a subject developing, or having already developed, preterm birth comprising, consisting of, or consisting essentially of: obtaining a biological sample from a subject; determining the expression level of one or more biomarkers that are associated with preterm birth in the biological sample; and comparing the expression level of the biomarkers in the biological sample with that of a control, wherein the presence of one or more of the biomarkers in the sample that is in an amount greater than that of the control indicates preterm birth.

Another aspect of the present disclosure provides a method determining the efficacy of an preterm birth treatment regime in a subject comprising, consisting of, or consisting essentially of: determining a baseline value for the expression of one or more biomarkers associated with preterm birth; administering to the subject a progesterone therapy regime; and redetermining the expression levels of one or more biomarkers in the subject, wherein observed decreases in one or more of the biomarker expression levels is correlated with the efficacy of the therapeutic regimen.

Another aspect of the present disclosure provides a composition of matter comprising, consisting of, or consisting essentially of: a probe array for determining a biomarker level in a sample, the array comprising of a plurality of probes that hybridizes to one or more biomarkers that are associated with preterm birth; or a kit for determining a biomarker level in a sample, comprising the probe array and instructions for carrying out the determination of biomarker expression level in the sample. In certain embodiments the probe array further comprises a solid support with the plurality of probes attached thereto.

In one embodiment, the biomarker comprises Progesterone Receptor Membrane Component 1 (PGRMC1).

In some embodiments, the subject is a mammal. In other embodiments, the subject is a human.

In other embodiments, the biological sample is selected from the group consisting of tissues, cells, biopsies, blood, lymph, serum, plasma, urine, saliva, mucus, vaginal secretions, cervical secretions, and tears. In certain embodiments, the sample comprises plasma.

One aspect of the present disclosure provides a method of determining the risk for preterm birth comprising: quantifying the amount of Progesterone Receptor Membrane Component 1 (PGRMC1) present in a biological sample derived from a pregnant subject; and predicting the subject as having an increased risk for preterm birth if the amount of the PGRMC1 biomarker is altered in the biological sample derived from the subject compared to a reference control.

One aspect of the present disclosure provides a method of diagnosing preterm birth comprising: quantifying the amount of Progesterone Receptor Membrane Component 1 (PGRMC1) present in a biological sample derived from a pregnant subject; and diagnosing the subject as having an increased risk for preterm birth if the amount of the PGRMC1 biomarker is altered in the biological sample derived from the subject compared to a reference control.

One aspect of the present disclosure provides a method for determining the efficacy of a preterm birth treatment comprising: determining a baseline value for the amount of Progesterone Receptor Membrane Component 1 (PGRMC1) present in a first biological sample comprising plasma derived from a pregnant subject; and determining a post-treatment value for the amount of PGRMC1 present in a second biological sample comprising plasma derived from the pregnant subject after the subject has been administered a treatment for the preterm birth, wherein an observed decrease in the value for the amount of PGRMC1 present in the post-treatment sample compared to the baseline value is correlated with the efficacy of the therapeutic regimen.

One aspect of the present disclosure provides a kit for diagnosing preterm birth comprising: a probe for determining a level of PGRMC1 biomarker for preterm birth in a biological sample derived from a pregnant subject; and instructions for carrying out the determination of the biomarker level in the biological sample and for diagnosing the subject as having preterm birth if the level of the PGRMC1 biomarker is greater in the biological sample derived from the subject compared to a reference control.

One aspect of the present disclosure provides a kit for determining the efficacy of a preterm birth treatment comprising: a probe specific for Progesterone Receptor Membrane Component 1 (PGRMC1) biomarker; and instructions for determining a baseline value for the amount of PGRMC1 present in a first biological sample derived from a pregnant subject and for determining a post-treatment value for the amount of PGRMC1 present in a second biological sample derived from the pregnant subject after the subject has been administered a treatment for the preterm birth, wherein an observed decrease in the value for the amount of PGRMC1 present in the post-treatment sample compared to the baseline value is correlated with the efficacy of the therapeutic regimen.

One aspect of the present disclosure provides a kit for determining the risk for preterm birth, the kit comprising: primers for amplification of a Progesterone Receptor Membrane Component 1 (PGRMC1) nucleic acid biomarker present in a biological sample derived from a pregnant subject; and instructions for quantifying the expression level of the PGRMC1 biomarker in the biological sample and for predicting the subject as having an increased risk for preterm birth if the expression level of the PGRMC1 biomarker is altered in the biological sample derived from the subject compared to a reference control.

One aspect of the present disclosure provides a kit for determining the efficacy of a preterm birth treatment comprising: primers for amplification of a Progesterone Receptor Membrane Component 1 (PGRMC1) nucleic acid biomarker; and instructions for determining a baseline value for the amount of PGRMC1 present in a first biological sample derived from a pregnant subject and for determining a post-treatment value for the amount of PGRMC1 present in a second biological sample derived from the pregnant subject after the subject has been administered a treatment for the preterm birth, wherein an observed decrease in the value for the amount of PGRMC1 present in the post-treatment sample compared to the baseline value is correlated with the efficacy of the therapeutic regimen.

### BRIEF DESCRIPTION OF THE DRAWINGS

The foregoing aspects and other features of the present disclosure are explained in the following description, taken in connection with the accompanying drawings, wherein:

FIGS. 1A-1B are A) Western blot and B) graph showing calcium-induced cytrotrophoblast apoptotic cell death reduction by progesterone (R5020) pretreatment.

FIGS. 2A-2C show immunohistochemical staining of fetal membrane sections probed with antibody for PGRMC1 expression in A) preterm no labor (PTNL) fetal membrane sections, B) term no labor (TNL) fetal membrane sections, and C) premature rupture of membranes (PPROM) fetal membrane sections.

FIG. 3 is a graph showing showing PGRMC1 expression levels in plasma determined by Western blot analysis for clinical groups: in term (Term), premature rupture of membranes (PPROM), and preterm labor (PTL).

FIGS. 4A-4C show the effect of knocking down PGRMC1 expression on the efficacy of medroxyprogesterone acetate (MPA) pretreatment in HTR8/SVneo cells. A) Western blot demonstrating significant reduction in PGRMC1 expression in HTR8/SVneo cells with PGRMC1-specific small interfering RNA (siRNA) treatment. B) A representative zymogram demonstrating that knocking down PGRMC1 expression reduces the efficacy of MPA pretreatment to inhibit tumor necrosis factor-α (TNF-α)-induced matrix metalloproteinase 9 (MMP-9) activity. C) A graph demonstrating that knocking down PGRMC1 expression reduces the efficacy of MPA pretreatment to inhibit TNF-α-induced matrix MMP-9 activity.

### DETAILED DESCRIPTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Currently, less than 35% patients who are identified as being at high risk based on obstetric history or cervical length will actually deliver preterm. Therefore the majority of these patients are receiving unnecessary therapies. The present disclosure provides a biomarker to identify women at risk for preterm birth and to identify individuals that may benefit from progesterone therapy.

The present disclosure provides the progesterone receptor called Progesterone Receptor Membrane Component 1 (PGRMC1) as a biomarker for preterm birth. Previous investigations evaluating PGRMC1 expression in blood were primarily focused on expression in peripheral leukocytes. While previous investigators failed to show a difference in expression during the menstrual cycle, a significant decrease was observed in PGRMC1 expression in subjects with premature ovarian failure and polycystic ovarian syndrome. [16, 17] Evidence exists that PGRMC1 expression is elevated in the setting of Crohn's disease when compared to ulcerative colitis. In addition, PGRMC1 in cancer is believed to promote tumorigenesis and plasma levels are significantly higher in patients with small cell lung cancer. [18]

First, the present inventors demonstrated PGRMC1 as playing an important role in the protection of cells of the fetal membranes from oxidative stress and inflammation induced cell death and activation of matrix metalloproteinases (MMP). Further, PGRMC1 protein levels in plasma were shown by the present inventors to be higher in subjects that deliver as a result of preterm labor even when those samples were collected remote from delivery. Methods and kits are provided herein for measuring PGRMC1 as a biomarker in pregnancy to predict individuals at risk for preterm delivery.

### DEFINITIONS

Articles “a” and “an” are used herein to refer to one or to more than one (i.e. at least one) of the grammatical object of the article. By way of example, “an element” means at least one element and can include more than one element.

Unless otherwise defined, all technical terms used herein have the same meaning as commonly understood by one of ordinary skill in the art to which this disclosure belongs.

As used herein, the term “biomarker” refers to a naturally occurring biological molecule present in a subject at varying concentrations useful in predicting the risk or incidence of a disease or a condition, such as preterm birth. For example, the biomarker can be a protein present in higher or lower amounts in a subject at risk for preterm birth. The biomarker can include nucleic acids, ribonucleic acids, or a polypeptide used as an indicator or marker for preterm birth subject. In some embodiments, the biomarker is a protein. In certain embodiments, the biomarker is progesterone receptor membrane component 1 (PGRMC1).

As used herein, the term “preterm labor” and “preterm birth” are used interchangeable and refer to either (1) the presence of uterine contractions of sufficient frequency and intensity to effect progressive effacement and dilation of cervix prior to term gestation (e.g., between 20 and 37 wks) and/or a premature birth that takes place more than 3 weeks before the scheduled due date.

“About” is used to provide flexibility to a numerical range endpoint by providing that a given value may be “slightly above” or “slightly below” the endpoint without affecting the desired result.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition. In certain embodiments, the treatment comprises progesterone therapy.

The term “effective amount” or “therapeutically effective amount” refers to an amount sufficient to effect beneficial or desirable biological and/or clinical results.

The present disclosure provides biomarkers useful for determining the risk of preterm birth in a subject. The present disclosure also provides methods of using such biomarker expression profiles to monitor a subject's response to treatment (e.g., efficacy of a treatment or therapy regimen) for conditions such as preterm birth.

Advantageously, the methods of the present disclosure are noninvasive, highly specific, and sensitive.

In one embodiment, the present disclosure profiles biomarkers found in the plasma for the diagnosis and prognosis of preterm birth.

In one embodiment, the present disclosure identifies plasma protein profiles as biomarkers for determining the risk of, prognosis of, and/or diagnosis of conditions such as preterm birth. The inventors have determined that certain biomarkers are directly involved in preterm birth, and their expression pattern in plasma can be associated with the pathophysiological status of preterm birth. It was discovered that these biomarker expression patterns in subjects at risk of preterm birth are distinctly different from that of normal controls.

One aspect of the present disclosure provides biomarkers useful for determining the risk of, prognosis of, and/or diagnosis of conditions such as preterm birth. In one embodiment, the present disclosure provides biomarkers that are differentially expressed, such as upregulated, down-regulated, or disregulated in a condition such as preterm birth, as compared to normal populations who do not have the condition, such as preterm birth.

In one embodiment, the biomarker comprises PGRMC1.

In some embodiments, the biomarkers are selected from one or more biomarkers that are up-regulated or over-expressed in a subject at risk for preterm birth. In some embodiments, the biomarker comprises PGRMC1, wherein the up-regulation or over-expression of the biomarker in the subject's biological sample, when compared to a control, indicates that the subject is at risk of preterm birth.

In some specific embodiments, the biomarkers are selected from one or more biomarkers up-regulated or over-expressed more than 50-fold, 40-fold, 30-fold, 20-fold, 15-fold, 10-fold, 9-fold, 8-fold, 7-fold, 6-fold, 5-fold, 4-fold, 3-fold, 2-fold, 1.9-fold, 1.8-fold, 1.7-fold, 1.6-fold, 1.5-fold, 1.4-fold, 1.3-fold, 1.2-fold, or 1.1-fold in a subject at risk of preterm birth, when compared to a control. In some embodiments, the biomarker comprises PGRMC1, wherein the up-regulation or over-expression of the biomarker in the subject's biological sample, when compared to a control, indicates that the subject is at risk of preterm birth.

In one embodiment, the present disclosure provides a method for assessing the risk of preterm birth in a a subject comprising, consisting of, or consisting essentially of: determining a biomarker expression profile (expression level) in a biological sample from the subject; characterizing the subject's biomarker profile; and comparing the subject's biomarker profile with the biomarker profile of a control from subjects not at risk of preterm birth; and administering an appropriate progesterone therapy if one or more of the biomarkers are expressed.

In another embodiment, the present disclosure provides a method for determining the risk of a subject developing a condition such as preterm birth comprising, consisting of, or consisting essentially of: determining a biomarker expression profile (expression level) in a biological sample from the subject; characterizing the subject's biomarker profile; and comparing the subject's biomarker profile with the biomarker profile of a control profile from subjects not at risk of preterm birth; and administering an appropriate prophylactic progesterone therapy if one or more of the biomarkers are expressed.

In yet another embodiment, the present disclosure provides a method for determining the prognosis of a subject developing, or having already developed, a condition such as preterm birth comprising, consisting of, or consisting essentially of: determining a biomarker expression profile (expression level) in a biological sample from the subject; characterizing the subject's biomarker profile; and comparing the subject's biomarker profile with the biomakrer profile of a control profile from subjects not at risk of preterm birth; and administering appropriate progesterone therapy or altering an already existing progesterone therapy if one or more of the biomarkers are expressed.

In one embodiment, the method further includes obtaining the biological sample from the subject. In one embodiment, the diagnosis and/or prognosis of a condition such as preterm birth can be determined by comparing the subjects biomarker profile to a reference biomarker profile, such as one that corresponds to biological samples obtained from a normal population that do not have a condition such as preterm birth, or that corresponds to biological samples obtained from a population that have a condition such as preterm birth. Optionally, the reference profile comprises multiple biomarker expression profiles, with each corresponding to a different stage of a condition such as preterm birth.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. Preferably, the subject is a human patient that is pregnant.

The term “biological sample” as used herein includes, but is not limited to, a sample containing tissues, cells, and/or biological fluids isolated from a subject. Examples of biological samples include, but are not limited to, tissues, cells, biopsies, blood, lymph, serum, plasma, urine, saliva, mucus and tears. In one embodiment, the biological sample is a blood sample (such as a plasma sample). A biological sample may be obtained directly from a subject (e.g., by blood or tissue sampling) or from a third party (e.g., received from an intermediary, such as a healthcare provider or lab technician).

In some embodiments, the present disclosure provides methods for diagnosing a condition such as preterm birth by characterizing a biomarker comprising PGRMC1. In some embodiments, the present disclosure provides methods for diagnosing conditions such as preterm birth by characterizing a biomarker comprising PGRMC1, wherein the up-regulation or over-expression of the biomarker, when compared to a control, indicates that the subject has a condition such as preterm birth.

In other embodiments, the present disclosure provides methods for determining the risk of a subject developing a condition such as preterm birth by characterizing a biomarker comprising PGRMC1. In some embodiments, the present disclosure provides methods for determining the risk of a subject developing a condition such as preterm birth by characterizing a biomarker comprising PGRMC1, wherein the up-regulation or over-expression of the biomarker, when compared to a control, indicates that the subject has a condition such as preterm birth.

In yet other embodiments, the present disclosure provides methods for determining the prognosis of a subject having a condition such as preterm birth by characterizing a biomarker comprising PGRMC1.

In some embodiments, the present disclosure provides methods for determining the prognosis of a subject having a condition such as preterm birth by characterizing a biomarker comprising PGRMC1, wherein the up-regulation or over-expression of the biomarker, when compared to a control, indicates that the subject has a condition such as preterm birth.

Another aspect of the present disclosure provides for methods for monitoring the treatment of conditions such as preterm birth.

In one embodiment, the method comprises a method of determining the efficacy of a preterm birth treatment regime (e.g., progesterone therapy) in a subject comprising, consisting of, or consisting essentially of: determining a baseline value for the expression of one or more biomarkers associated with preterm birth; administering to the subject an preterm birth therapy regime; and redetermining the expression levels of one or more biomarkers in the subject, wherein observed decreases in one or more or the biomarker expression levels is correlated with the efficacy of the therapeutic regimen.

In instances where a decrease in the biomarker expression is not seen, a change in treatment may be warranted. Such a determination, and the different type of treatment to employ, can be made readily determined by one skilled in the art.

Another aspect of the present disclosure provides a composition of matter comprising, consisting of, or consisting essentially of: a probe array for determining a biomarker level in a sample, the array comprising of a plurality of probes that hybridizes to one or more biomarkers that are associated with preterm birth; or a kit for determining a biomaker level in a sample, comprising the probe array and instructions for carrying out the determination of biomarker expression level in the sample. In certain embodiments the probe array further comprises a solid support with the plurality of probes attached thereto.

The present disclosure provides a method of determining the risk of, prognosis of, and/or diagnosis of a condition such as preterm birth on at least one sample obtained from an individual. The individual may be any mammal, but is preferably a human.

In one aspect, the present disclosure provides a method of determining the risk for preterm birth comprising: quantifying the amount of Progesterone Receptor Membrane Component 1 (PGRMC1) present in a biological sample derived from a pregnant subject; and predicting the subject as having an increased risk for preterm birth if the amount of the PGRMC1 biomarker is altered in the biological sample derived from the subject compared to a reference control. In the method the biological sample can be selected from the group consisting of tissues, cells, biopsies, blood, lymph, serum, plasma, urine, saliva, mucus, vaginal secretions, cervical secretions, and tears.

In one embodiment of the method for determining the risk for preterm birth, the biological sample can comprise plasma and the amount of the PGRMC1 biomarker can be greater in the plasma derived from the subject compared to the reference control for predicting the subject as having an increased risk for preterm birth. In one embodiment, the biological sample can consist of plasma and the amount of the PGRMC1 biomarker can be greater in the plasma derived from the subject compared to the reference control for predicting the subject as having an increased risk for preterm birth.

In one embodiment of the method for determining the risk for preterm birth, the PGRMC1 can be a polypeptide and the quantifying can be carried out by an assay comprising one or a combination of Western Blotting, Array system, affinity matrice, and immunoassay. The immunoassay can comprise one or a combination of immunocytochemistry, immunohistochemistry, competitive binding assay, a non-competitive assay, a sandwich assay, a fluoroimmunoassay, an immunofluorometric assay, an immunoradiometric assay, a luminescence assay, and a chemiluniescence assay. The immunoassay can comprise one or a combination of an antibody and a binding fragment. The antibody or binding fragment can be coupled to a solid phase.

In one embodiment of the method for determining the risk for preterm birth, the PGRMC1 can be a polypeptide and the quantifying can be carried out by mass spectroscopy.

In one embodiment of the method for determining the risk for preterm birth, the PGRMC1 can be a nucleic acid and the quantifying can be carried out by one or a combination of Polymerase Chain Reaction, Real Time-Polymerase Chain Reaction, Real Time Reverse Transcriptase-Polymerase Chain Reaction, Northern blot analysis, in situ hybridization, and Array system.

In one embodiment of the method for determining the risk for preterm birth, the subject can be a mammal. In one embodiment, the subject can be a human. The subject can be a human and the biological sample can be derived from the subject prior to 24 weeks of gestation. The subject can be a human and the biological sample can be derived from the subject at about 28 weeks of gestation.

In one embodiment, the method for determining the risk for preterm birth can further comprise administering an appropriate prophylactic progesterone therapy if the subject is predicted as having an increased risk for preterm birth.

In one aspect, the present disclosure provides a method of diagnosing preterm birth comprising: quantifying the amount of Progesterone Receptor Membrane Component 1 (PGRMC1) present in a biological sample derived from a pregnant subject; and diagnosing the subject as having an increased risk for preterm birth if the amount of the PGRMC1 biomarker is altered in the biological sample derived from the subject compared to a reference control. In the method, the biological sample can be selected from the group consisting of tissues, cells, biopsies, blood, lymph, serum, plasma, urine, saliva, mucus, vaginal secretions, cervical secretions, and tears.

In one embodiment of the method for diagnosing preterm birth, the biological sample can comprise plasma and the amount of the PGRMC1 biomarker can be greater in the plasma derived from the subject compared to the reference control for diagnosing the subject as having an increased risk for preterm birth. In one embodiment, the biological sample can consist of plasma and the amount of the PGRMC1 biomarker can be greater in the plasma derived from the subject compared to the reference control for diagnosing the subject as having an increased risk for preterm birth.

In one embodiment of the method for diagnosing preterm birth, the PGRMC1 can be a polypeptide and the quantifying can be carried out by an assay comprising one or a combination of Western Blotting, Array system, affinity matrice, and immunoassay. The immunoassay can comprise one or a combination of immunocytochemistry, immunohistochemistry, competitive binding assay, a non-competitive assay, a sandwich assay, a fluoroimmunoassay, an immunofluorometric assay, an immunoradiometric assay, a luminescence assay, and a chemiluniescence assay. The immunoassay can comprise one or a combination of an antibody and a binding fragment. The antibody or binding fragment can be coupled to a solid phase.

In one embodiment of the method for diagnosing preterm birth, the PGRMC1 can be a polypeptide and the quantifying can be carried out by mass spectroscopy.

In one embodiment of the method for diagnosing preterm birth, the PGRMC1 can be a nucleic acid and the quantifying can be carried out by one or a combination of Polymerase Chain Reaction, Real Time-Polymerase Chain Reaction, Real Time Reverse Transcriptase-Polymerase Chain Reaction, Northern blot analysis, in situ hybridization, and Array system.

In one embodiment of the method for diagnosing preterm birth, the subject can be a mammal. In one embodiment, the subject can be a human. The subject can be a human and the biological sample can be derived from the subject prior to 24 weeks of gestation. The subject can be a human and the biological sample can be derived from the subject at about 28 weeks of gestation.

In one embodiment of the method for diagnosing preterm birth, the method can further comprise administering an appropriate prophylactic progesterone therapy if the subject is predicted as having an increased risk for preterm birth.

In one aspect, the present disclosure provides a method for determining the efficacy of a preterm birth treatment comprising: determining a baseline value for the amount of Progesterone Receptor Membrane Component 1 (PGRMC1) present in a first biological sample comprising plasma derived from a pregnant subject; and determining a post-treatment value for the amount of PGRMC1 present in a second biological sample comprising plasma derived from the pregnant subject after the subject has been administered a treatment for the preterm birth, wherein an observed decrease in the value for the amount of PGRMC1 present in the post-treatment sample compared to the baseline value is correlated with the efficacy of the therapeutic regimen.

In one embodiment of the method for determining the efficacy of a preterm birth treatment, the PGRMC1 can be a polypeptide and the quantifying can be carried out by an assay comprising one or a combination of Western Blotting, Array system, affinity matrice, and immunoassay. The immunoassay can comprise one or a combination of immunocytochemistry, immunohistochemistry, competitive binding assay, a non-competitive assay, a sandwich assay, a fluoroimmunoassay, an immunofluorometric assay, an immunoradiometric assay, a luminescence assay, and a chemiluniescence assay. The immunoassay can comprise one or a combination of an antibody and a binding fragment. The antibody or binding fragment can be coupled to a solid phase.

In one embodiment of the method for determining the efficacy of a preterm birth treatment, the PGRMC1 can be a polypeptide and the quantifying can be carried out by mass spectroscopy.

In one embodiment of the method for determining the efficacy of a preterm birth treatment, the PGRMC1 can be a nucleic acid and the quantifying can be carried out by one or a combination of Polymerase Chain Reaction, Real Time-Polymerase Chain Reaction, Real Time Reverse Transcriptase-Polymerase Chain Reaction, Northern blot analysis, in situ hybridization, and Array system.

In one embodiment of the method for determining the efficacy of a preterm birth treatment, the biological sample can be selected from the group consisting of tissues, cells, biopsies, blood, lymph, serum, plasma, urine, saliva, mucus, vaginal secretions, cervical secretions, and tears. The biological sample can comprise plasma. The biological sample can consist of plasma. The subject can be a mammal. The subject can be a human.

In one aspect, the present disclosure provides a kit for determining the risk for preterm birth, the kit comprising: a probe for determining a level of PGRMC1 biomarker for preterm birth in a biological sample derived from a pregnant subject; and instructions for carrying out the determination of the biomarker level in the biological sample and for predicting the subject as having an increased risk for preterm birth if the level of the PGRMC1 biomarker is greater in the biological sample derived from the subject compared to a reference control.

In one embodiment of the kit for determining the risk for preterm birth, the PGRMC1 biomarker can be a polypeptide and the probe can comprise an antibody or a binding fragment specific for PGRMC1 polypeptide. The determination of the PGRMC1 biomarker level can be carried out by an assay comprising one or a combination of Western Blotting, affinity matrice, and immunoassay. The immunoassay can comprise one or a combination of immunocytochemistry, immunohistochemistry, competitive binding assay, a non-competitive binding assay, a sandwich assay, a fluoroimmunoassay, an immunofluorometric assay, an immunoradiometric assay, a luminescence assay, and a chemiluniescence assay.

In one embodiment of the kit for determining the risk for preterm birth, the PGRMC1 biomarker can be a nucleic acid and the probe can comprises a nucleic acid specific for hybridization to PGRMC1 nucleic acid. The determination of the PGRMC1 biomarker level can be carried out by one or a combination of Polymerase Chain Reaction, Real Time-Polymerase Chain Reaction, Real Time Reverse Transcriptase-Polymerase Chain Reaction, Northern blot analysis, in situ hybridization, and Array system. The probe can be attached to a solid support.

In one embodiment of the kit for determining the risk for preterm birth, the kit can further comprise reagents for determining the level of the PGRMC1 biomarker.

In one embodiment of the kit for determining the risk for preterm birth, the biological sample can be selected from the group consisting of tissues, cells, biopsies, blood, lymph, serum, plasma, urine, saliva, mucus, vaginal secretions, cervical secretions, and tears. The biological sample can comprise plasma. The biological sample can consist of plasma. The subject can be a mammal. The subject can be a human. The subject can be a human and the biological sample can be derived from the subject prior to 24 weeks of gestation. The subject can be a human and the biological sample can be derived from the subject at about 28 weeks of gestation.

In one aspect, the present disclosure provides a kit for diagnosing preterm birth comprising: a probe for determining a level of PGRMC1 biomarker for preterm birth in a biological sample derived from a pregnant subject; and instructions for carrying out the determination of the biomarker level in the biological sample and for diagnosing the subject as having preterm birth if the level of the PGRMC1 biomarker is greater in the biological sample derived from the subject compared to a reference control.

In one embodiment of the kit for diagnosing preterm birth, the PGRMC1 biomarker can be a polypeptide and the probe can comprise an antibody or a binding fragment specific for PGRMC1 polypeptide. The determination of the PGRMC1 biomarker level can be carried out by an assay comprising one or a combination of Western Blotting, affinity matrice, and immunoassay. The immunoassay can comprise one or a combination of immunocytochemistry, immunohistochemistry, competitive binding assay, a non-competitive binding assay, a sandwich assay, a fluoroimmunoassay, an immunofluorometric assay, an immunoradiometric assay, a luminescence assay, and a chemiluniescence assay.

In one embodiment of the kit for diagnosing preterm birth, the PGRMC1 biomarker can be a nucleic acid and the probe can comprise a nucleic acid specific for hybridization to PGRMC1 nucleic acid. The determination of the PGRMC1 biomarker level can be carried out by one or a combination of Polymerase Chain Reaction, Real Time-Polymerase Chain Reaction, Real Time Reverse Transcriptase-Polymerase Chain Reaction, Northern blot analysis, in situ hybridization, and Array system. The probe can be attached to a solid support.

In one embodiment of the kit for diagnosing preterm birth, the kit can further comprise reagents for determining the level of the PGRMC1 biomarker.

In one embodiment of the kit for diagnosing preterm birth, the biological sample can be selected from the group consisting of tissues, cells, biopsies, blood, lymph, serum, plasma, urine, saliva, mucus, and tears. The biological sample can comprise plasma. The subject can be a mammal. The subject can be a human. The subject can be a human and the biological sample can be derived from the subject prior to 24 weeks of gestation. The subject can be a human and the biological sample can be derived from the subject at about 28 weeks of gestation.

In one aspect, the present disclosure provides a kit for determining the efficacy of a preterm birth treatment comprising: a probe specific for Progesterone Receptor Membrane Component 1 (PGRMC1) biomarker; and instructions for determining a baseline value for the amount of PGRMC1 present in a first biological sample derived from a pregnant subject and for determining a post-treatment value for the amount of PGRMC1 present in a second biological sample derived from the pregnant subject after the subject has been administered a treatment for the preterm birth, wherein an observed decrease in the value for the amount of PGRMC1 present in the post-treatment sample compared to the baseline value is correlated with the efficacy of the therapeutic regimen.

In one embodiment of the kit for determining the efficacy of a preterm birth treatment, the PGRMC1 biomarker can be a polypeptide and the probe can comprise an antibody or a binding fragment specific for PGRMC1 polypeptide. The determination of the PGRMC1 biomarker level can be carried out by an assay comprising one or a combination of Western Blotting, affinity matrice, and immunoassay. The immunoassay can comprise one or a combination of immunocytochemistry, immunohistochemistry, competitive binding assay, a non-competitive binding assay, a sandwich assay, a fluoroimmunoas say, an immunofluorometric assay, an immunoradiometric assay, a luminescence assay, and a chemiluniescence assay.

In one embodiment of the kit for determining the efficacy of a preterm birth treatment, the PGRMC1 biomarker can be a nucleic acid and the probe can comprise a nucleic acid specific for hybridization to PGRMC1 nucleic acid. The determination of the PGRMC1 biomarker level can be carried out by one or a combination of Polymerase Chain Reaction, Real Time-Polymerase Chain Reaction, Real Time Reverse Transcriptase-Polymerase Chain Reaction, Northern blot analysis, in situ hybridization, and Array system. The probe can be attached to a solid support.

In one embodiment of the kit for determining the efficacy of a preterm birth treatment, the kit can further comprise reagents for determining the level of the PGRMC1 biomarker.

In one embodiment of the kit for determining the efficacy of a preterm birth treatment, the biological sample can be selected from the group consisting of tissues, cells, biopsies, blood, lymph, serum, plasma, urine, saliva, mucus, vaginal secretions, cervical secretions, and tears. The biological sample can comprise plasma. The biological sample can consist of plasma. The subject can be a mammal. The subject can be a human.

In one aspect, the present disclosure provides a kit for determining the risk for preterm birth, the kit comprising: primers for amplification of a Progesterone Receptor Membrane Component 1 (PGRMC1) nucleic acid biomarker present in a biological sample derived from a pregnant subject; and instructions for quantifying the expression level of the PGRMC1 biomarker in the biological sample and for predicting the subject as having an increased risk for preterm birth if the expression level of the PGRMC1 biomarker is altered in the biological sample derived from the subject compared to a reference control.

In one embodiment of the kit for determining the risk for preterm birth, the quantification of the PGRMC1 biomarker expression level can comprise one or a combination of Polymerase Chain Reaction, Real Time-Polymerase Chain Reaction, Real Time Reverse Transcriptase-Polymerase Chain Reaction, Northern blot analysis, in situ hybridization, and Array system.

In one embodiment of the kit for determining the risk for preterm birth, the kit can further comprise one or more hybridization probes specific for the PGRMC1 biomarker. The probe can be attached to a solid support.

In one embodiment of the kit for determining the risk for preterm birth, the kit can further comprise reagents for determining the level of the PGRMC1 biomarker.

In one embodiment of the kit for determining the risk for preterm birth, the biological sample can be selected from the group consisting of tissues, cells, biopsies, blood, lymph, serum, plasma, urine, saliva, mucus, vaginal secretions, cervical secretions, and tears. The biological sample can comprise plasma. The biological sample can consist of plasma. The subject can be a mammal. The subject can be a human.

In one aspect, the present disclosure provides a kit for determining the efficacy of a preterm birth treatment comprising: primers for amplification of a Progesterone Receptor Membrane Component 1 (PGRMC1) nucleic acid biomarker; and instructions for determining a baseline value for the amount of PGRMC1 present in a first biological sample derived from a pregnant subject and for determining a post-treatment value for the amount of PGRMC1 present in a second biological sample derived from the pregnant subject after the subject has been administered a treatment for the preterm birth, wherein an observed decrease in the value for the amount of PGRMC1 present in the post-treatment sample compared to the baseline value is correlated with the efficacy of the therapeutic regimen.

In one embodiment of the kit for determining the efficacy of a preterm birth treatment, the determination of the PGRMC1 biomarker level can comprise one or a combination of Polymerase Chain Reaction, Real Time-Polymerase Chain Reaction, Real Time Reverse Transcriptase-Polymerase Chain Reaction, Northern blot analysis, in situ hybridization, and Array system.

In one embodiment of the kit for determining the efficacy of a preterm birth treatment, the kit can further comprise one or more hybridization probes specific for the PGRMC1 biomarker. The probe can be attached to a solid support.

In one embodiment of the kit for determining the efficacy of a preterm birth treatment, the kit can further comprise reagents for determining the level of the PGRMC1 biomarker.

In one embodiment of the kit for determining the efficacy of a preterm birth treatment, the biological sample can be selected from the group consisting of tissues, cells, biopsies, blood, lymph, serum, plasma, urine, saliva, mucus, vaginal secretions, cervical secretions, and tears. The biological sample can comprise plasma. The biological sample can consist of plasma. The subject can be a mammal. The subject can be a human.

The present disclosure may involve obtaining more than one sample, such as two samples, such as three samples, four samples or more from individuals, and preferably the same individual. This allows the relative comparison of expression of the biomarker between the multiple samples. Alternatively, a single sample may be compared against a “standardized” sample, such a sample comprising material or data from several samples, preferably also from several individuals.

Before analyzing the sample, it will often be desirable to perform one or more sample preparation operations upon the sample. Typically, these sample preparation operations will include such manipulations as concentration, suspension, extraction of intracellular material, e.g., protein or nucleic acids from tissue/whole cell samples and the like, amplification of nucleic acids, fragmentation, transcription, labelling and/or extension reactions.

Any method required for the processing of a sample prior to detection by any of the methods noted herein falls within the scope of the present disclosure. These methods are typically well known by a person skilled in the art.

It is within the general scope of the present disclosure to provide methods for the detection of protein biomarker. An aspect of the present disclosure relates to the detection of the proteins as described in the plots and graphs of the figures contained herein. As used herein, the term “detect” or “determine the presence of” refers to the qualitative measurement of undetectable, low, normal, or high concentrations of one or more biomarkers such as, for example, polypeptides or nucleic acids and other biological molecules. Detection may include 1) detection in the sense of presence versus absence of one or more biomarkers as well as 2) the registration/quantification of the level or degree of expression of one or more biomarkers, depending on the method of detection employed. The term “quantify” or “quantification” may be used interchangeable, and refer to a process of determining the quantity or abundance of a substance in a sample (e.., a biomarker), whether relative or absolute. For example, quantification may be determined by methods including but not limited to, band intensity on a Northern or Western blot, micro-array analysis, qRT-PCR, or by various other methods known in the art.

The detection of one or more biomarker molecules allows for the classification, diagnosis and prognosis of a condition such as preterm birth. The classification of such conditions is of relevance both medically and scientifically and may provide important information useful for the diagnosis, prognosis and treatment of the condition. The diagnosis of a condition such as preterm birth is the affirmation of the presence of the condition, as is the object of the present disclosure, on the expression of at least one biomarker herein. Prognosis is the estimate or prediction of the probable outcome of a condition such as preterm birth and the prognosis of such is greatly facilitated by increasing the amount of information on the particular condition.

The kit of claim 94, wherein the subject is a human.

Any method of detection falls within the general scope of the present disclosure. The detection methods may be generic for the detection of polypeptides, nucleic acids, and the like. The detection methods may be directed towards the scoring of a presence or absence of one or more biomarker molecules or may be useful in the detection of expression levels.

The detection methods can be divided into two categories herein referred to as in situ methods or screening methods. The term in situ method refers to the detection of protein molecules and/or nucleic acid in a sample wherein the structure of the sample has been preserved. This may thus be a biopsy wherein the structure of the tissue is preserved. In situ methods are generally histological i.e. microscopic in nature and include but are not limited to methods such as: cytochemistry, immunocytochemistry, immunohistochemistry, in situ hybridization techniques, and in situ PCR methods.

Screening methods generally employ techniques of molecular biology and most often require the preparation of the sample material in order to access the nucleic acid and/or polypeptide molecules to be detected. Screening methods include, but are not limited to methods such as: Western Blotting, Array systems, affinity matrices, Northern blotting and PCR techniques, such as real-time quantitative RT-PCR.

One aspect of the present disclosure is to provide a probe which can be used for the detection of a nucleic acid and/or polypeptide molecule as defined herein. A probe as defined herein is a specific sequence of a nucleic acid and/or polypeptide used to detect nucleic acids and/or polypeptides by binding and/or hybridization. For example, a nucleic acid is also here any nucleic acid, natural or synthetic such as DNA, RNA, LNA or PNA. A probe may be labeled, tagged or immobilized or otherwise modified according to the requirements of the detection method chosen. A label or a tag is an entity making it possible to identify a compound to which it is associated. It is within the scope of the present disclosure to employ probes that are labeled or tagged by any means known in the art such as but not limited to: radioactive labeling, fluorescent labeling and enzymatic labeling. Furthermore the probe, labeled or not, may be immobilized or attached to a solid support to facilitate detection according to the detection method of choice and this may be accomplished according to the preferred method of the particular detection method.

Another aspect of the present disclosure regards the detection of nucleic acid and/or polypeptide molecules by any method known in the art. In the following are given examples of various detection methods that can be employed for this purpose, and the present disclosure includes all the mentioned methods, but is not limited to any of these.

In situ hybridization (ISH) applies and extrapolates the technology of nucleic acid and/or polypeptide hybridization to the single cell level, and, in combination with the art of cytochemistry, immunocytochemistry and immunohistochemistry, permits the maintenance of morphology and the identification of cellular markers to be maintained and identified, allows the localization of sequences to specific cells within populations, such as tissues and blood samples. ISH is a type of hybridization that uses a complementary nucleic acid to localize one or more specific nucleic acid sequences in a portion or section of tissue (in situ), or, if the tissue is small enough, in the entire tissue (whole mount ISH). DNA ISH can be used to determine the structure of chromosomes and the localization of individual genes and optionally their copy numbers. Fluorescent DNA ISH (FISH) can for example be used in medical diagnostics to assess chromosomal integrity. RNA ISH is used to assay expression and gene expression patterns in a tissue/across cells, such as the expression of miRNAs/nucleic acid molecules. Sample cells are treated to increase their permeability to allow the probe to enter the cells, the probe is added to the treated cells, allowed to hybridize at pertinent temperature, and then excess probe is washed away. A complementary probe is labeled with a radioactive, fluorescent or antigenic tag, so that the probe's location and quantity in the tissue can be determined using autoradiography, fluorescence microscopy or immunoassay, respectively. The sample may be any sample as herein described. The probe is likewise a probe according to any probe based upon the biomarkers mentioned herein.

An aspect of the present disclosure includes the method of detection by in situ hybridization as described herein.

In situ PCR is the PCR based amplification of the target nucleic acid sequences prior to ISH. For detection of RNA, an intracellular reverse transcription (RT) step is introduced to generate complementary DNA from RNA templates prior to in situ PCR. This enables detection of low copy RNA sequences.

Prior to in situ PCR, cells or tissue samples are fixed and permeabilized to preserve morphology and permit access of the PCR reagents to the intracellular sequences to be amplified. PCR amplification of target sequences is next performed either in intact cells held in suspension or directly in cytocentrifuge preparations or tissue sections on glass slides. In the former approach, fixed cells suspended in the PCR reaction mixture are thermally cycled using conventional thermal cyclers. After PCR the cells are cytocentrifugated onto glass slides with visualization of intracellular PCR products by ISH or immunohistochemistry. In situ PCR on glass slides is performed by overlaying the samples with the PCR mixture under a coverslip which is then sealed to prevent evaporation of the reaction mixture. Thermal cycling is achieved by placing the glass slides either directly on top of the heating block of a conventional or specially designed thermal cycler or by using thermal cycling ovens. Detection of intracellular PCR-products is achieved by one of two entirely different techniques. In indirect in situ PCR by ISH with PCR-product specific probes, or in direct in situ PCR without ISH through direct detection of labeled nucleotides (e.g. digoxigenin-11-dUTP, fluorescein-dUTP, ³H-CTP or biotin-16-dUTP) which have been incorporated into the PCR products during thermal cycling.

An embodiment of the present disclosure concerns the method of in situ PCR as mentioned herein above for the detection of nucleic acid molecules as detailed herein.

A microarray is a microscopic, ordered array of nucleic acids, proteins, small molecules, cells or other substances that enables parallel analysis of complex biochemical samples. A DNA microarray consists of different nucleic acid probes, known as capture probes that are chemically attached to a solid substrate, which can be a microchip, a glass slide or a microsphere-sized bead. Microarrays can be used e.g. to measure the expression levels of large numbers of polypeptides/proteins/nucleic acids simultaneously.

Microarrays can be fabricated using a variety of technologies, including printing with fine-pointed pins onto glass slides, photolithography using pre-made masks, photolithography using dynamic micromirror devices, ink-jet printing, or electrochemistry on microelectrode arrays.

An aspect of the present disclosure regards the use of microarrays for the expression profiling of biomarkers in conditions such as preterm birth. For this purpose, and by way of example, RNA is extracted from a cell or tissue sample, the small RNAs (18-26-nucleotide RNAs) are size-selected from total RNA using denaturing polyacrylamide gel electrophoresis (PAGE). Then oligonucleotide linkers are attached to the 5′ and 3′ ends of the small RNAs and the resulting ligation products are used as templates for an RT-PCR reaction with 10 cycles of amplification. The sense strand PCR primer has a Cy3 fluorophore attached to its 5′ end, thereby fluorescently labelling the sense strand of the PCR product. The PCR product is denatured and then hybridized to the microarray. A PCR product, referred to as the target nucleic acid that is complementary to the corresponding RNA capture probe sequence on the array will hybridize, via base pairing, to the spot at which the capture probes are affixed. The spot will then fluoresce when excited using a microarray laser scanner. The fluorescence intensity of each spot is then evaluated in terms of the number of copies of a particular biomarker, using a number of positive and negative controls and array data normalization methods, which will result in assessment of the level of expression of a particular biomarker.

Several types of microarrays can be employed such as spotted oligonucleotide microarrays, pre-fabricated oligonucleotide microarrays or spotted long oligonucleotide arrays.

In spotted oligonucleotide microarrays the capture probes are oligonucleotides complementary to nucleic acid sequences. This type of array is typically hybridized with amplified PCR products of size-selected small RNAs from two samples to be compared that are labelled with two different fluorophores. Alternatively, total RNA containing the small RNA fraction is extracted from the abovementioned two samples and used directly without size-selection of small RNAs, and 3′ end labeled using T4 RNA ligase and short RNA linkers labelled with two different fluorophores. The samples can be mixed and hybridized to one single microarray that is then scanned, allowing the visualization of up-regulated and down-regulated biomarker genes in one go. The downside of this is that the absolute levels of gene expression cannot be observed, but the cost of the experiment is reduced by half. Alternatively, a universal reference can be used, comprising of a large set of fluorophore-labelled oligonucleotides, complementary to the array capture probes.

In pre-fabricated oligonucleotide microarrays or single-channel microarrays, the probes are designed to match the sequences of known or predicted biomarkers. There are commercially available designs that cover complete genomes from companies such as Affymetrix, or Agilent. These microarrays give estimations of the absolute value of gene expression and therefore the comparison of two conditions requires the use of two separate microarrays.

Spotted long oligonucleotide arrays are composed of 50 to 70-mer oligonucleotide capture probes, and are produced by either ink-jet or robotic printing. Short Oligonucleotide Arrays are composed of 20-25-mer oligonucleotide probes, and are produced by photolithographic synthesis (AFFYMETRIX) or by robotic printing. More recently, Maskless Array Synthesis from NIMBLEGEN SYSTEMS has combined flexibility with large numbers of probes. Arrays can contain up to 390,000 spots, from a custom array design.

An embodiment of the present disclosure concerns the method of microarray use and analysis as described herein.

The terms “PCR reaction”, “PCR amplification”, “PCR”, “pre-PCR”, “Q-PCR”, “real-time quantitative PCR” and “real-time quantitative RT-PCR” are interchangeable terms used to signify use of a nucleic acid amplification system, which multiplies the target nucleic acids being detected. Examples of such systems include the polymerase chain reaction (PCR) system and the ligase chain reaction (LCR) system. Other methods recently described and known to the person of skill in the art are the nucleic acid sequence based amplification and Q Beta Replicase systems. The products formed by said amplification reaction may or may not be monitored in real time or only after the reaction as an end-point measurement.

Real-time quantitative RT-PCR is a modification of polymerase chain reaction used to rapidly measure the quantity of a product of polymerase chain reaction. It is preferably done in real-time, thus it is an indirect method for quantitatively measuring starting amounts of DNA, complementary DNA or ribonucleic acid (RNA). This is commonly used for the purpose of determining whether a genetic sequence is present or not, and if it is present the number of copies in the sample. There are 3 methods which vary in difficulty and detail. Like other forms of polymerase chain reaction, the process is used to amplify DNA samples, using thermal cycling and a thermostable DNA polymerase.

The three commonly used methods of quantitative polymerase chain reaction are through agarose gel electrophoresis, the use of SYBR Green, a double stranded DNA dye, and the fluorescent reporter probe. The latter two of these three can be analysed in real-time, constituting real-time polymerase chain reaction method.

Agarose gel electrophoresis is the simplest method, but also often slow and less accurate then other methods, depending on the running of an agarose gel via electrophoresis. It cannot give results in real time. The unknown sample and a known sample are prepared with a known concentration of a similarly sized section of target DNA for amplification. Both reactions are run for the same length of time in identical conditions (preferably using the same primers, or at least primers of similar annealing temperatures). Agarose gel electrophoresis is used to separate the products of the reaction from their original DNA and spare primers. The relative quantities of the known and unknown samples are measured to determine the quantity of the unknown. This method is generally used as a simple measure of whether the probe target sequences are present or not, and rarely as ‘true’ Q-PCR.

Using SYBR Green dye is more accurate than the gel method, and gives results in real time. A DNA binding dye binds all newly synthesized double stranded (ds)DNA and an increase in fluorescence intensity is measured, thus allowing initial concentrations to be determined. However, SYBR Green will label all dsDNA including any unexpected PCR products as well as primer dimers, leading to potential complications and artefacts. The reaction is prepared as usual, with the addition of fluorescent dsDNA dye. The reaction is run, and the levels of fluorescence are monitored; the dye only fluoresces when bound to the dsDNA. With reference to a standard sample or a standard curve, the dsDNA concentration in the PCR can be determined.

The fluorescent reporter probe method is the most accurate and most reliable of the methods. It uses a sequence-specific nucleic acid based probe so as to only quantify the probe sequence and not all double stranded DNA. It is commonly carried out with DNA based probes with a fluorescent reporter and a quencher held in adjacent positions, so-called dual-labelled probes. The close proximity of the reporter to the quencher prevents its fluorescence; it is only on the breakdown of the probe that the fluorescence is detected. This process depends on the 5′ to 3′ exonuclease activity of the polymerase involved. The real-time quantitative PCR reaction is prepared with the addition of the dual-labelled probe. On denaturation of the double-stranded DNA template, the probe is able to bind to its complementary sequence in the region of interest of the template DNA (as the primers will too). When the PCR reaction mixture is heated to activate the polymerase, the polymerase starts synthesizing the complementary strand to the primed single stranded template DNA. As the polymerisation continues it reaches the probe bound to its complementary sequence, which is then hydrolysed due to the 5′-3′ exonuclease activity of the polymerase thereby separating the fluorescent reporter and the quencher molecules. This results in an increase in fluorescence, which is detected. During thermal cycling of the real-time PCR reaction, the increase in fluorescence, as released from the hydrolysed dual-labelled probe in each PCR cycle is monitored, which allows accurate determination of the final, and so initial, quantities of DNA.

Any method of PCR that can determine the expression of a nucleic acid molecule as defined herein falls within the scope of the present disclosure. A preferred embodiment of the present disclosure includes the real-time quantitative RT-PCR method, based on the use of either SYBR Green dye or a dual-labelled probe for the detection and quantification of nucleic acids according to the herein described.

An aspect of the present disclosure includes the detection of the nucleic acid molecules herein disclosed by techniques such as Northern blot analysis. Many variations of the protocol exist.

The following examples are offered by way of illustration and not by way of limitation.

### EXAMPLES

### Example 1

### Progesterone Anti-Inflammatory and Anti-Apoptotic Effects Driven Through PGRMC1 Receptor

Progesterone Receptor Membrane Component 1 (PGRMC1) is highly expressed in primary chorion cells of the fetal membranes. Controversy remains over the presence of canonical progestin nuclear receptors in fetal chorion cells of the membranes. [13-15] For the present study, Western blot analysis of proteins extracted from cultured HTR8/SVneo cells was performed and a first trimester cytotrophoblast cell line demonstrated PGRMC1 with a molecular mass of 22 kDa, and no detectable PR-A and PR-B (PgR1294; DAKO, Carpiniteria, CA) (data not shown). These data suggest that the effects mediated by progesterone treatment of the chorion may occur primarily through the PGRMC1 receptor. Immunohistochemistry of fetal membranes for PGRMC1 corroborated the Western blot data, demonstrating robust staining in the chorion and luminal side of the amnion (data not shown).

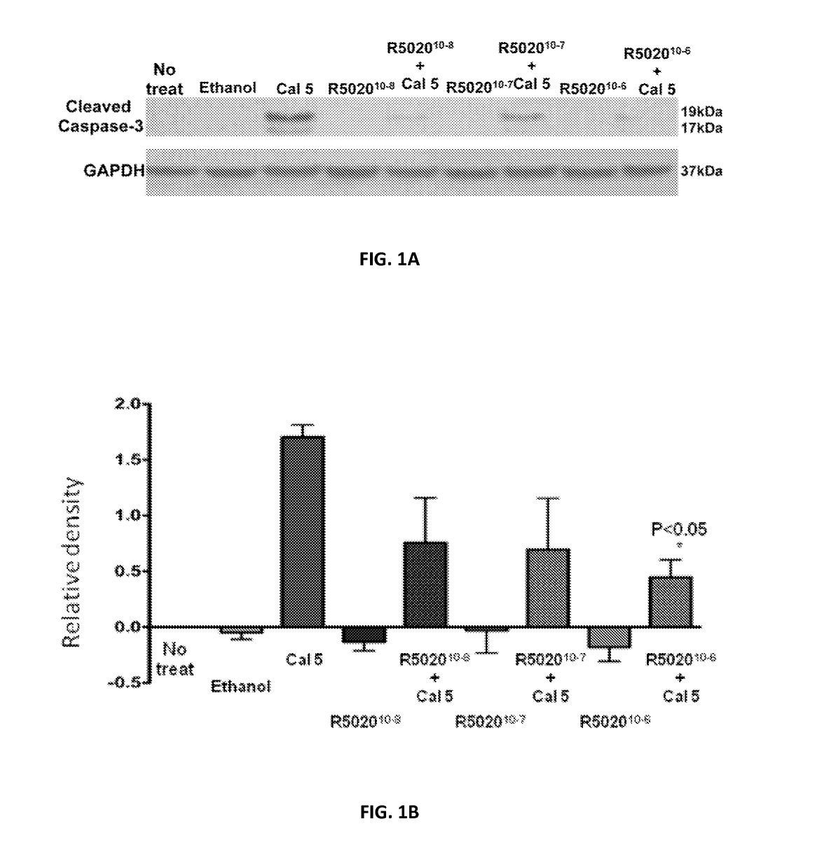

In addition, it was determined that pretreatment of HTR8/Sv cells with progestin R5020 (known to have high binding to PGRMC1) protected the cells against the calcium-ionophore calcimycin-induced apoptotic cell death as quantified using the CELLTITER 96 Aqueous Non-Radioactive Cell Proliferation Assay (PROMEGA; see FIG. 1). In separate studies, it was also found that R5020 treatment resulted in lower caspase 3 cleavage following treatment with calcimycin (data not shown). Progesterone treatment also blunted TNF α-mediated induction of metalloproteinase 9 (MMP-9) activity (data not shown). Together, these data indicate that progesterone has strong anti-inflammatory and anti-apoptotic effects driven primarily through the non-cannonical PGRMC1 receptor in fetal membranes.

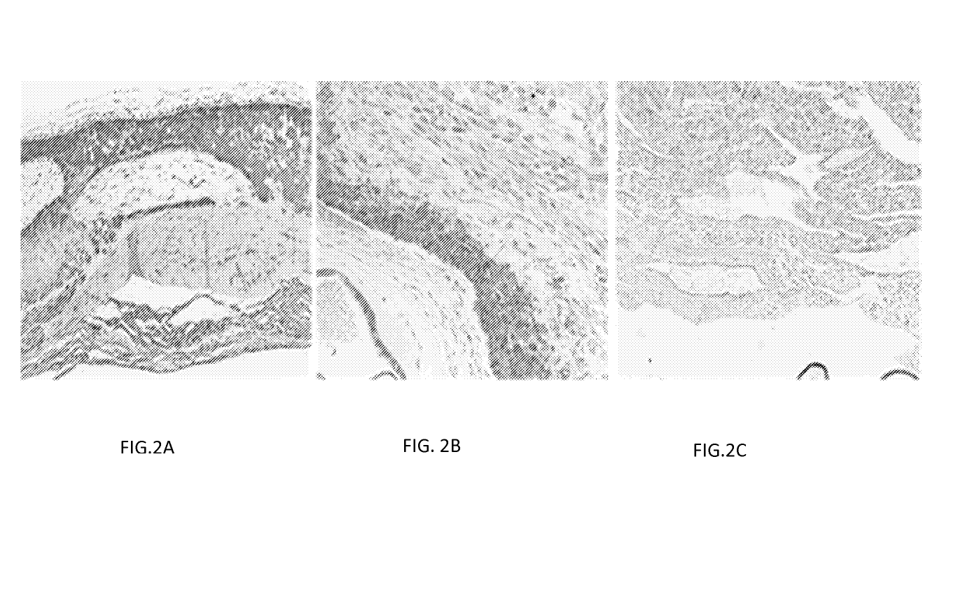

Using immunohistochemistry, PGRMC1 expression was characterized in the fetal membranes from preterm no labor (PTNL) subjects, term no labor (TNL) subjects, and premature rupture of membranes (PPROM) subjects. Representative images from each clinical group are shown in FIG. 2. PGRMC1 expression was significantly lower in PPROM fetal membrane tissue compared to TNL or PTNL (see Table 1 below).

TABLE 1
Adjusted Ordinal logistic regression

| | Denominator:Numerator | | |
|---|---|---|---|
| Predictor | of Odds Ratio (OR) | OR (95% Cl) | P value |
| Clinical | Chorion cells:Decidua cells | 1.64 (1.4-1.9) | <0.0001 |
| phenotype | Amnion cells:Decidua cells | 4.71 (3.9-5.7) | 0.0001 |
| | Amnion cells:Chorion cells | 1.42 (1.35-1.59 | 0.0001 |
| Cell type | TNL:PPROM | 1.35 (1.15-1.59) | <0.0001 |
| | PTNL:PPROM | 4.66 (3.84-5.65) | <0.0001 |
| | PTNL:TNL | 3.45 (2.89-4.12) | <0.0001 |

Data presented as OR (95% Cl) after controlling for reader, measurements, cell layer, site of collection (rupture vs. distant) and clinical group

The frequency of distribution of PGRMC1 was calculated for each clinical group described for FIG. 2 and demonstrated that PGRMC1 staining in the PPROM group was more likely to have a lower score (1 and 2) when compared to TNL and PTNL (PPROM vs. TNL vs. PTNL: 38.8% vs. 13.8% vs. 2.4%, respectively).

### Example 2

### Quantification of PGRMC1 Protein in Maternal Plasma as a Predictor of Preterm Birth

In this experiment, PGRMC1 protein was quantified in a set of maternal plasma samples that included preterm labor (PTL) subjects, preterm premature rupture of membranes (PPROM) subjects, and term delivery subjects. In PPROM subjects, plasma was collected after rupture of membranes but remote from delivery. In PTL subjects, plasma was collected when the subject was admitted to Labor and Delivery for symptomatic contractions. Ultimately delivery was delayed an average of 18 days in these subjects. In term subjects' plasma was collected upon admission to Labor and Delivery either for elective cesarean delivery or early labor. PGRMC1 expression was determined by Western blot and quantified with densitometry according to the methods described below.

Plasma samples were thawed and centrifuged at 13,000 g for 10 minutes, and the supernatant was diluted 1:1 in 20 mM Tris, pH 7.4 followed by incubation with 200 μl of Separopore blue CL-6B (BIO-WORLD, Dublin, Ohio) slurry (equilibrated in 20 mM Tris, pH 7.4) for 30 min. The slurry was then clarified by centrifugation at 1,000 g for 3 minutes and 10 μl of the supernatant was mixed with loading buffer (INVITROGEN, Carlsbad, Calif.) and subjected to PAGE using 4-12% Bis-Tris NuPAGE precast gels (INVITROGEN). The separated proteins were transferred to PVDF membranes and blocked with 5% milk in 1×Tris buffered saline-Tween 20 (TBS-T) buffer for 1 h at room temperature. The blots were probed with primary antibody at 4° C. overnight and secondary antibodies at room temperature for 1 h with washing in between, and the latter detected using the enhanced chemiluminescence system (AMERSHAM) in accordance with the manufacturer's protocol.

The following anti-human antibodies were used according to the manufacturer's specifications: PGRMC1 antibody (SIGMA) was used at 1:2000 dilution. Secondary goat anti-rabbit IgG HRP-linked antibody (CELL SIGNALING TECHNOLOGY) was used at 1:2000 dilution. The density of each band was measured by IMAGE J analysis software (NIH) and data presented as relative intensity. Three replicates of Western blotting were performed for each sample. Purified recombinant PGRMC1 protein was used as a standard for quantification of unknown samples. A control sample was loaded onto all gels as an internal quality control.

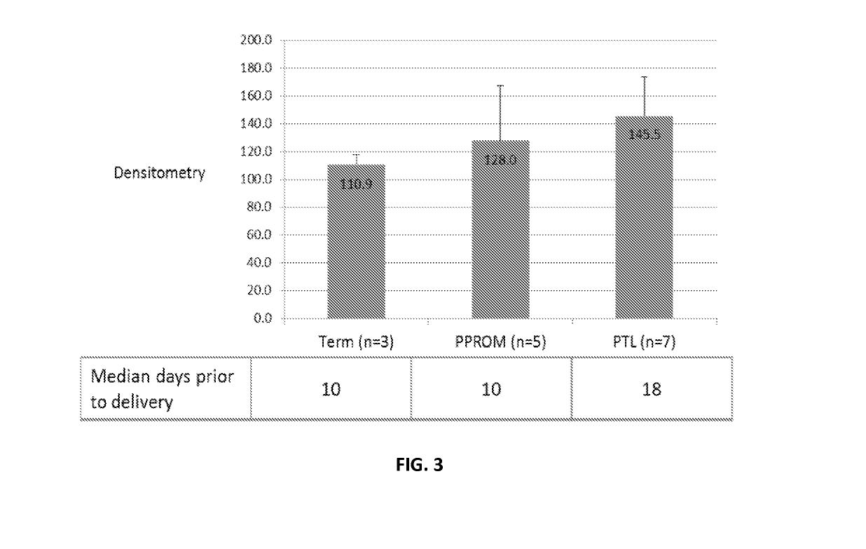

The data are shown in FIG. 3. PGRMC1 protein level varied by clinical phenotype (see FIG. 5). Specifically, the amount of PGRMC1 protein in the Term clinical group was lower than that observed in the PPROM amd PTL groups.

### Example 3

### PGRMC1 siRNA Effect on Cytokine Induced MMP 9 Zymography

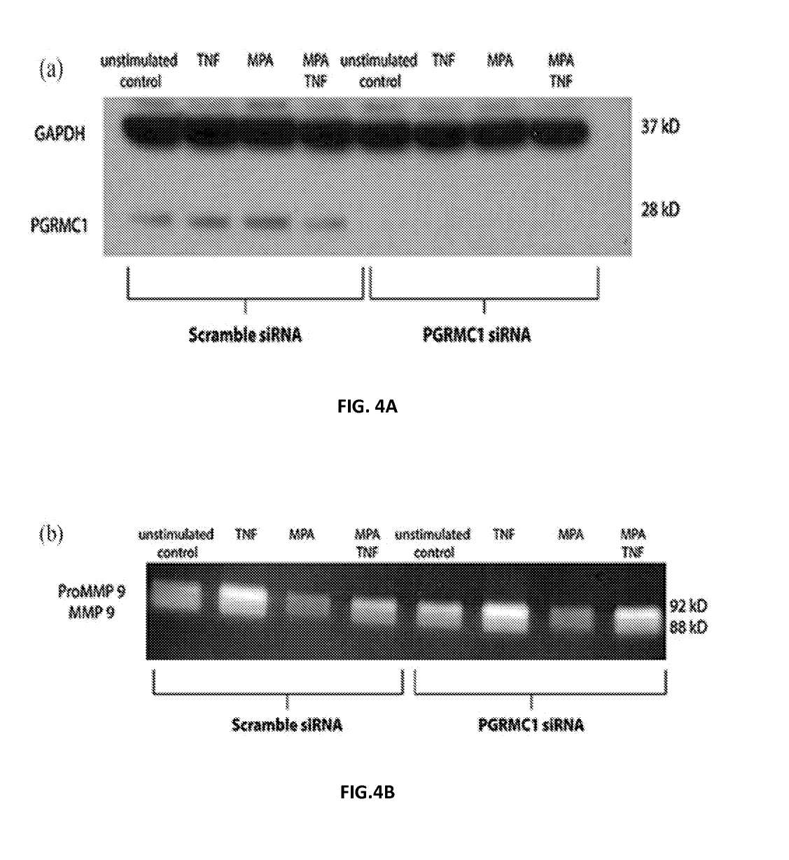

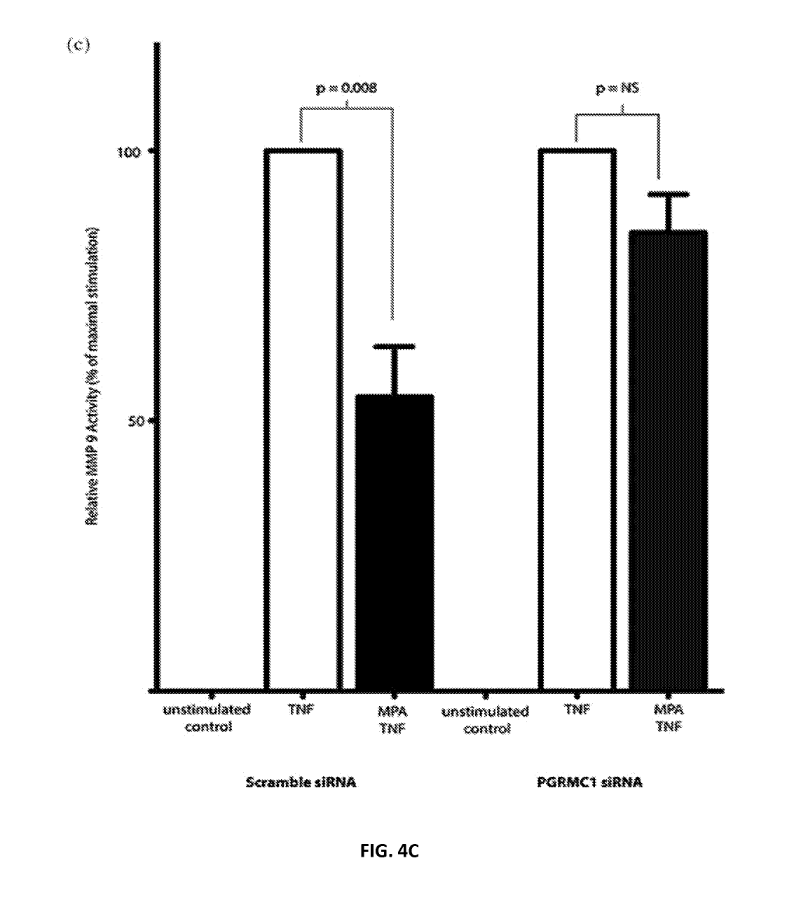

Experiments were performed to investigate the effect of reducing PGRMC1 expression in HTR8/SVneo cells. First, Western blot analysis was performed to confirm the effect on PGRMC1 expression in HTR8/SVneo cells treated with PGRMC1-specific small interfering RNA (siRNA). A scramble siRNA was used as a control. The Western blot analysis confirmed that PGRMC1 expression was significantly reduced (at least >50%) when treated with PGRMC1 siRNA compared to controls (see FIG. 4A).

The results showed that knocking down PGRMC1 expression reduced the efficacy of medroxyprogesterone acetate (MPA) pretreatment to inhibit tumor necrosis factor-α (TNF-α)-induced Matrix Metalloprotease 9 (MMP-9) activity in cells pre-treated with PGRMC1 siRNA. The inhibitory effect of MPA pre-treatment on TNFα-induced activity was diminished when compared to cells treated with the scramble siRNA group (see FIGS. 4B&4C). Specifically, MPA pre-treatment reduced TNFα-induced MMP-9 activity by 45% when compared to the stimulated controls (p<0.008). However, in the PGRMC1 siRNA group this effect was lost with MPA pre-treatment reducing MMP-9 activity to a lesser degree when compared with stimulated controls (15%, p=0.100). Both the unstimulated (vehicle) control and the MPA groups are shown in the zymogram. The relative MMP-9 activity represents activity in excess of baseline for each experimental group. The unstimulated (vehicle) control only is represented graphically for both siRNA treatments. Experiments were replicated on 5 separate occasions. Data are mean+standard error of the mean.

Any patents or publications mentioned in this specification are indicative of the levels of those skilled in the art to which the invention pertains. These patents and publications are herein incorporated by reference to the same extent as if each individual publication was specifically and individually indicated to be incorporated by reference.

One skilled in the art will readily appreciate that the present invention is well adapted to carry out the objects and obtain the ends and advantages mentioned, as well as those inherent therein. The present examples along with the methods described herein are presently representative of preferred embodiments, are exemplary, and are not intended as limitations on the scope of the invention. Changes therein and other uses will occur to those skilled in the art which are encompassed within the spirit of the invention as defined by the scope of the claims.

### REFERENCES

1. Birth, C.o.U.P. and A.H. Outcomes, Preterm Birth: Causes, Consequences, and Prevention, ed. R. E. Behrman and A. S. Butler. 2007: The National Academies Press.
2. Goldenberg, R. L., et al., Epidemiology and causes of preterm birth. Lancet, 2008. 371(9606): p. 75-84.
3. Swamy Gk, Ø.T. S. R., ASsociation of preterm birth with long-term survival, reproduction, and next-generation preterm birth. JAMA: The Journal of the American Medical Association, 2008. 299(12): p. 1429-1436.
4. Menon, R., et al., Biomarkers of spontaneous preterm birth: an overview of the literature in the last four decades. Reprod Sci, 2011. 18(11): p. 1046-70.
5. Berghella, V., et al., Fetal fibronectin testing for reducing the risk of preterm birth. Cochrane Database Syst Rev, 2008(4): p. CD006843.
6. Sanchez-Ramos, L., et al., Fetal fibronectin as a short-term predictor of preterm birth in symptomatic patients: a meta-analysis. Obstet Gynecol, 2009. 114(3): p. 631-40.
7. Jams, J. D., et al., The Length of the Cervix and the Risk of Spontaneous Premature Delivery. New England Journal of Medicine, 1996. 334(9): p. 567-573.
8. Parry, S., et al., Universal maternal cervical length screening during the second trimester: pros and cons of a strategy to identify women at risk of spontaneous preterm delivery. American Journal of Obstetrics and Gynecology, 2012. 207(2): p. 101-106.
9. Werner, E. F., et al., Universal cervical-length screening to prevent preterm birth: a cost-effectiveness analysis. Ultrasound in Obstetrics & Gynecology, 2011. 38(1): p. 32-37.
10. Meisl, D., M. Hubler, and S. Arnold, [Treatment of fibroepithelial hyperplasia (FEH) of the mammary gland in the cat with the progesterone antagonist Aglepristone (Alizine)]. Schweiz Arch Tierheilkd, 2003. 145(3): p. 130-6.
11. Fonseca, E. B., et al., Progesterone and the risk of preterm birth among women with a short cervix. N Engl J Med, 2007. 357(5): p. 462-9.
12. Hassan, S. S., et al., Vaginal progesterone reduces the rate of preterm birth in women with a sonographic short cervix: a multicenter, randomized, double-blind, placebo-controlled trial. Ultrasound Obstet Gynecol, 2011. 38(1): p. 18-31.
13. Haluska, G. J., et al., Progesterone receptor localization and isoforms in myometrium, decidua, and fetal membranes from rhesus macaques: evidence for functional progesterone withdrawal at parturition. J Soc Gynecol Investig, 2002. 9(3): p. 125-36.
14. Merlino, A., et al., Nuclear progesterone receptor expression in the human fetal membranes and decidua at term before and after labor. Reprod Sci, 2009. 16(4): p. 357-63.
15. Mills, A. A., et al., Characterization of progesterone receptor isoform expression in fetal membranes. Am J Obstet Gynecol, 2006. 195(4): p. 998-1003.
16. Mansouri, M. R., et al., Alterations in the expression, structure and function of progesterone receptor membrane component-1 (PGRMC1) in premature ovarian failure. Hum Mol Genet, 2008. 17(23): p. 3776-83.
17. Schuster, J., et al., Down-regulation of progesterone receptor membrane component 1 (PGRMC1) in peripheral nucleated blood cells associated with premature ovarian failure (POF) and polycystic ovary syndrome (PCOS). Reprod Biol Endocrinol, 2010. 8: p. 58.
18. Mir, S. U., et al., Elevated progesterone receptor membrane component 1/sigma-2 receptor levels in lung tumors and plasma from lung cancer patients. Int J Cancer, 2012. 131(2): p. E1-9.

What is claimed is:

1.-14. (canceled)

15. A method of diagnosing preterm birth comprising:
quantifying the amount of Progesterone Receptor Membrane Component 1 (PGRMC1) present in a biological sample derived from a pregnant subject, wherein
the subject is indicated as having an increased risk for preterm birth if the amount of the PGRMC1 biomarker is altered in the biological sample derived from the subject compared to a reference control.

16. The method of claim 15, wherein the biological sample is selected from the group consisting of tissues, cells, biopsies, blood, lymph, serum, plasma, urine, saliva, mucus, and tears.

17. The method of claim 16, wherein the biological sample comprises plasma and the amount of the PGRMC1 biomarker is greater in the plasma derived from the subject compared to the reference control.

18. The method of claim 15, wherein the PGRMC1 is a polypeptide and the quantifying is carried out by an assay comprising one or a combination of Western Blotting, Array system, affinity matrice, and immunoassay.

19.-25. (canceled)

26. The method of claim 15, wherein the biological sample is derived from the subject prior to 24 weeks of gestation.

27. The method of claim 15, wherein the biological sample is derived from the subject at about 28 weeks of gestation.

28. The method of claim 15, further comprising administering an appropriate prophylactic progesterone therapy if the subject is predicted as having an increased risk for preterm birth.

29. A method for determining the efficacy of a preterm birth treatment comprising:
determining a baseline value for the amount of Progesterone Receptor Membrane Component 1 (PGRMC1) present in a first biological sample comprising plasma derived from a pregnant subject; and
determining a post-treatment value for the amount of PGRMC1 present in a second biological sample comprising plasma derived from the pregnant subject after the subject has been administered a treatment for the preterm birth, wherein an observed decrease in the value for the amount of PGRMC1 present in the post-treatment sample compared to the baseline value is correlated with the efficacy of the therapeutic regimen.

30. The method of claim 29, wherein the biological sample is selected from the group consisting of tissues, cells, biopsies, blood, lymph, serum, plasma, urine, saliva, mucus, vaginal secretions, cervical secretions, and tears.

31. The method of claim 29, wherein the biological sample comprises plasma.

32. The method of claim 29, wherein the PGRMC1 is a polypeptide and the quantifying is carried out by an assay comprising one or a combination of Western Blotting, Array system, affinity matrice, and immunoassay.

33.-53. (canceled)

54. A kit for diagnosing preterm birth comprising:
i) a probe comprising an antibody or a binding fragment specific for PGRMC1 polypeptide for determining a level of PGRMC1 biomarker for preterm birth in a plasma sample derived from a pregnant subject;
ii) instructions for carrying out the determination of the biomarker level in the biological sample and for diagnosing the subject as having preterm birth if the level of the PGRMC1 biomarker is greater in the biological sample derived from the subject compared to a reference control; and
iii) reagents for determining the level of the PGRMC1 biomarker in the plasma sample.

55. (canceled)

56. The kit of claim 54, wherein the determination of the PGRMC1 biomarker level is carried out by an assay comprising one or a combination of Western Blotting, affinity matrice, and immunoassay.

57. The kit of claim 56, wherein the immunoassay comprises one or a combination of immunocytochemistry, immunohistochemistry, competitive binding assay, a non-competitive binding assay, a sandwich assay, a fluoroimmunoassay, an immunofluorometric assay, an immunoradiometric assay, a luminescence assay, and a chemiluniescence assay.

58. (canceled)

59. (canceled)

60. The kit of claim 54, wherein the probe is attached to a solid support.

61.-65. (canceled)

66. The kit of claim 54, wherein the biological sample is derived from the subject prior to 24 weeks of gestation.

67. The kit of claim 54, wherein the biological sample is derived from the subject at about 28 weeks of gestation.

68-97. (canceled)
